(19) United States
(12) Patent Application Publication
(10) Pub. No.: US 2022/0022460 A1
(43) Pub. Date: Jan. 27, 2022

(54) USE OF GUAR DERIVATIVES IN BIOFUNGICIDE COMPOSITIONS

(71) Applicant: RHODIA OPERATIONS, Aubervilliers (FR)

(72) Inventors: Jean-Christophe CASTAING, Sèvres (FR); Florence LAMBERT, Paris (FR); Mirjana GELO-PUJIC, Sérézin-du-Rhône (FR)

(21) Appl. No.: 17/298,310

(22) PCT Filed: Nov. 29, 2019
(86) PCT No.: PCT/EP2019/083105

§ 371 Date: May 28, 2021

Related U.S. Application Data
(60) Provisional application No. 62/772,846, filed on Nov. 29, 2018.

(51) Int. Cl. A01N 63/20; A01N 63/22; A01N 63/38; A01N 63/30; A01N 63/23; A01N 63/27

(52) U.S. Cl. CPC A01N 63/20; A01N 63/22; A01N 63/27; A01N 63/30; A01N 63/23; A01N 63/38

(57) ABSTRACT

The present invention relates to a fungicide composition comprising a biofungicide and a non-derivatized guar gum.

This application claims priority to U.S. P A No. 62/772846 filed on Nov. 29, 2018, the whole content of this application being incorporated herein by reference for all purposes.

The present invention concerns the use of guar derivatives in bio fungicide compositions, especially the use of guar derivatives for the growth of microorganisms, in particular of suppressive microorganisms.

Diseases caused by fungal species are considered among the most widespread and damaging of plants worldwide. Presently, control of plant fungal diseases is largely dependent upon the application of certain chemicals. Although some of these chemicals are known to have negative environmental and human health problems, nevertheless such chemical agents continue to be in wide use due to their strong activity against important fungal diseases, and limited availability of environmentally safer and effective alternatives.

Generally, biological control of diseases commonly infecting plants in the root zone (rhizosphere) and the leaf zone (phylloplane) are preferred over more traditional synthetic chemical control methodologies. Such biocontrol agents usually cause little or no injury to the plant host or the environment, and some may even favor normal plant development. However, most such biocontrol organisms are typically very limited either in the scope of their effectiveness against fungal diseases, or in their ability to survive and maintain the activity under formulation conditions, storage conditions, application under practical field conditions and during treatment applications.

Attempts have been made to control plant fungal diseases by using certain microorganisms.

Microorganisms used in agriculture are generally bacteria, yeasts, molds, mycorrhizae.

There is thus a need to find means to maintain or even improve the activity and efficiency of target microorganisms.

There is also a need to find means to specifically maintain or improve the growth of a target microorganism in a given medium.

More generally there is a need to provide formulations useful to selectively improve the growth of target microorganism in a given medium.

There is also a need to provide formulations useful to selectively stimulate a target microorganism in a given medium.

The inventors have found that a specific guar derivative was useful to address these needs.

Therefore, the present invention relates to fungicide composition comprising a bio-fungicide and a guar derivative, preferably a non-derivatized guar gum.

According to the invention, the guar derivative of the invention may increase the growth rate of the bio-fungicide.

The growth rate of microorganisms, in particular of suppressive microorganism, may be measured by the following method:

Microorganisms are incubated in a culture media in presence of guar. Sampling is performed at different times in order to determine the number of colony forming unit (CFU) using the spread-plating method. With this methodology, the evolution of the number of bacterial cells (expressed as CFU) as a function of time is obtained. The microorganism growth follows an exponential law: Nₜ=N₀e^{(μt)} with μ the growth rate of microorganisms. The value of the growth rate of microorganisms μ is obtained by fitting the experimental data in logarithmic scale, it corresponds to the slope of the evolution of ln(Nₜ) as a function of time (linear plot: ln(Nₜ)=ln(N₀)+μt).

According to an embodiment, the present invention relates to method for maintaining or increasing the growth rate of microorganisms, in particular of suppressive microorganism, comprising a step of contacting said microorganism with a guar derivative of the invention which is preferably a non-derivatized guar gum.

The present invention is based on the use of a guar derivative of the invention which enables to maintain and keep constant over the time the bio fungicide effect of microorganisms, in particular of suppressive microorganism, and in other words to maintain the growth rate of microorganisms, and in particular to maintain the bacterial growth rate.

Advantageously, the use of said guar derivative enables to increase the biofungicidal activity of microorganisms, in particular of suppressive microorganism, in other words the bio fungicidal activity of a microorganism is significantly or substantially greater when applied in association or combination with a guar derivative of the invention than that obtained without the use of said guar derivative.

Preferably, according to the invention, when using the guar derivative as defined above, the growth rate of microorganisms is increased of at least 5%, preferably of at least 10%, in comparison to the growth rate of microorganisms when no guar derivative is used.

The present invention also relates to use of a microorganism, in particular of a suppressive microorganism, and of a guar derivative which is preferably a non-derivatized guar gum, as biofungicide.

The present invention also relates to a kit comprising at least one microorganism, in particular a suppressive microorganism, and at least one guar derivative which is preferably a non-derivatized guar gum, as well as to the use of said kit as bio fungicide.

According to an embodiment, the present invention relates to a method for controlling fungal organism comprising applying a fungicide composition as described previously.

The present invention also relates to a method of controling or preventing infection of a plant by phytopathogenic fungi, comprising the step of applying to said plant a fungicide composition as described previously.

According to an embodiment, the guar derivative as mentioned above is used in a plant, on a seed or in the soil.

Throughout the description, including the claims, the term “comprising one” or “comprising a” should be understood as being synonymous with the term “comprising at least one”, unless otherwise specified, “between” and “from . . . to . . . ” should be understood as being inclusive of the limits.

As used herein, “weight percent,” “wt %,” “percent by weight,” “% by weight,” and variations thereof refer to the concentration of a substance as the weight of that substance divided by the total weight of the composition and multiplied by 100.

Should the disclosure of any patents, patent applications, and publications which are incorporated herein by reference conflict with the description of the present application to the extent that it may render a term unclear, the present description shall take precedence.

### Guars

Processes for making derivatives of guar gum splits are generally known. Typically, guar splits are reacted with one or more derivatizing agents under appropriate reaction conditions to produce a guar polysaccharide having the desired substituent groups. Suitable derivatizing reagents are commercially available and typically contain a reactive functional group, such as an epoxy group, a chiorohydrin group, or an ethylenically unsaturated group, and at least one other substituent group, such as a cationic, nonionic or anionic substituent group, or a precursor of such a substituent group per molecule, wherein substituent group may be linked to the reactive functional group of the derivatizing agent by bivalent linking group, such as an alkylene or oxyalkylene group. Suitable cationic substituent groups include primary, secondary, or tertiary amino groups or quaternary ammonium, sulfonium, or phosphinium groups. Suitable nonionic substituent groups include hydroxyalkyl groups, such as hydroxypropyl groups. Suitable anionic groups include carboxyalkyl groups, such as carboxymethyl groups. The cationic, nonionic and/or anionic substituent groups may be introduced to the guar polysaccharide chains via a series of reactions or by simultaneous reactions with the respective appropriate derivatizing agents.

The guar may be treated with a crosslinking agent, such for example, borax (sodium tetra borate) is commonly used as a processing aid in the reaction step of the water-splits process to partially crosslink the surface of the guar splits and thereby reduces the amount of water absorbed by the guar splits during processing. Other crosslinkers, such as, for example, glyoxal or titanate compounds, are known.

In one embodiment, the guar derivative of the composition of the present invention is a non-derivatized guar derivative, more typically a non-derivatized guar gum.

In one embodiment, the guar derivative is a derivatized guar derivative that is substituted at one or more sites of the polysaccharide with a substituent group that is independently selected for each site from the group consisting of cationic substituent groups, nonionic substituent groups, and anionic substituent groups.

In one embodiment, the guar derivative of the composition of the present invention is derivatized guar derivative, more typically a derivatized guar. Suitable derivatized guars include, for example, hydroxypropyl trimethylammonium guar, hydroxypropyl lauryldimethylammonium guar, hydroxypropyl stearyldimethylammonium guar, hydroxypropyl guar, carboxymethyl guar, guar with hydroxypropyl groups and hydroxypropyl trimethylammonium groups, guar with carboxymethyl hydroxypropyl groups and mixtures thereof.

The amount of derivatizing groups in a derivatized polysaccharide polymer may be characterized by the degree of substitution of the derivatized polysaccharide polymer or the molar substitution of the derivatized polysaccharide polymer.

As used herein, the terminology “degree of substitution” in reference to a given type of derivatizing group and a given polysaccharide polymer means the number of the average number of such derivatizing groups attached to each monomeric unit of the polysaccharide polymer. In one embodiment, the derivatized guar derivative exhibits a total degree of substitution (“DST”) of from about 0.001 to about 3.0, wherein:

DST is the sum of the DS for cationic substituent groups (“DScationic”), the DS for nonionic substituent groups (“DSnonionic”) and the DS for anionic substituent groups (“DSanionic”),

DScationic is from 0 to about 3, more typically from about 0.001 to about 2.0, and even more typically from about 0.001 to about 1.0,

DSnonionic is from 0 to 3.0, more typically from about 0.001 to about 2.5, and even more typically from about 0.001 to about 1.0, and

DSanionic is from 0 to 3.0, more typically from about 0.001 to about 2.0. As used herein, the term “molar substitution” or “ms” refers to the number of moles of derivatizing groups per moles of monosaccharide units of the guar. The molar substitution can be determined by the Zeisel-GC method. The molar substitution utilized by the present invention is typically in the range of from about 0.001 to about 3.

According to a preferred embodiment, the guar derivative of the composition of the present invention is a non-derivatized guar derivative, more typically a non-derivatized guar gum.

In the present application, guar designates the plant Cyanopsis tetragonoloba. Cyamopsis tetragonoloba (guar) gums as defined above may be used in a composition.

In the present application, “guar seeds” designates seeds derived from guar. Guar seeds comprise the hull, which is more or less fibrous, the germ, and two “guar splits” or “endosperm halves”, which constitute the endosperm of guar. The splits (or endosperm) is/are rich in galactomannans. The guar seeds generally consist of 35 to 40% by weight of endosperm, 42 to 47% by weight of germ, and 14 to 17% by weight of hull.

In the present application, “guar flour” or “guar powder” designates a powder derived from the guar endosperm.

In the present application, “native guar” designates macromolecular chains of the galactomannan type, derived from guar endosperm, not having been subjected to chemical modification by the grafting of chemical groups. Native guar comprises macromolecules containing a principal chain of D-mannopyranose units linked in the beta (1-4) position substituted by D-galactopyranose units in the beta (1-6) position. Native guar has a mannose/galactose ratio of about 2.

In the present application, “Cyamopsis tetragonoloba (guar) gum guar” (also referred to as “non-derivatized guar gum” or even “guar gum”) designates a product substantially consisting of native guar, in the form of guar splits, or of guar flour or powder.

As used herein, the “average molecular weight” of the guar gum means the weight average molecular weight of said guar gum.

According to anyone of the invention embodiments, the guar gum of the invention may have an average molecular weight (Mw) of between 2,000 Daltons and 5,000,000 Dalton. In one embodiment, the guar gum of the invention may have an average molecular weight (Mw) of between 100,000 Daltons and 4,500,000 Daltons, for instance between 500,000 Daltons and 4,000,000 Daltons, for instance between 1,000,000 Daltons and 3,500,000 Daltons, for instance between 2,000,000 and 3,500,000 Daltons.

In another embodiment, the guar gum of the invention may have an average molecular weight (Mw) of between about 2,000 Daltons and 90,000 Daltons, for instance between about 5,000 Daltons and 60,000 Daltons, for instance between about 5,000 Daltons and 40,000 Daltons, for instance between about 8,000 Daltons and 30,000 Daltons.

The average molecular weight of the guar gum may be measured by GPC (Gel Permeation Chromatography). Measurements may be carried out for instance using Shodex OH Pak columns and Agilent Refractive Index Detector.

The guar derivatives as defined above may be used in a composition.

The composition containing the guar derivative may be a solid or a liquid composition. In the case wherein the composition is solid, the composition may be in the form of a powder, a particle, an agglomerate, a flake, a granule, a pellet, a tablet, a brick, a paste, a block such as a molded block, a unit dose, or another solid form known to those of skill in the art. Preferably, the solid composition is in the form of a powder or a granule.

In some aspects, the composition containing the guar is in the form of a granule. Granules containing the guar derivative may be prepared in a three-step procedure: wet granulation followed by drying and sieving. The wet granulation step notably involves introduction and mixing of guar derivative powders and a carrier, and optionally other ingredients, in granulation equipment (such as a mixing granulator). The mixing is conducted with spraying of water to the mixture. The wet granulation step will yield wet granules containing the guar derivatives. The weight ratio between the carrier and the guar derivative which are to be mixed may be between 20:1 to 1:1, preferably, between 20:1 to 10:1. The water content introduced may be comprised between 10 wt % to 50 wt % based on the total weight of the wet granules. The carrier may be silicon dioxide, amorphous silica, precipitated silica, hydrated amorphous silica, precipitated silica, hydrated amorphous synthetic calcium silicate, hydrofobized precipitated silica, silica gel, sodium aluminium silicate, clay, zeolite, bentonite, layered silicate, caolim, sodium carbonate, sodium bicarbonate, sodium sulfate, sodium tripolyphosphate, sodium chloride, sodium silicate (water glass), magnesium chloride, calcium chloride, ammonium chloride, magnesium sulfate, calcium carbonate, calcium oxide, and/or calcium sulphate, or a mixture thereof. Notably, the carrier is selected from calcium chloride and calcium carbonate. The drying step notably involves drying the wet granules by using hot air flow. This step can usually be conducted in a fluid bed equipped with an air inlet and an air outlet. The sieving step may be conducted by using a vibrating plate.

The granules may have a diameter of 0.1 to 6 mm. Generally, normal granules have a diameter of 2-6 mm and micro granules have a diameter of 0.1-2 mm. Preferably, micro granules having a diameter of 0.5-1.6 mm are used.

Alternatively, the granules containing the guar derivative may be prepared by using extrusion methods well known by a person skilled in the art. The extrusion methods are described in U.S. Pat. No. 6,146,570. For example, the guar derivative and the carrier, and optionally other ingredients, may be blended with heating. The weight ratio between the carrier and the guar derivative may be between 20:1 to 1:1. Then a binder may be melted and introduced into the mixture of the guar derivative and the carrier. Then, an extrusion step may be carried out with extruder temperature maintained between 55° C. and 65° C. The soft warm granules may be formed and may be subsequently cooled below solidification point of the molten binder (at room temperature for instance) in order to obtain solid granules.

In the case that the composition (seed treatment composition or composition for foliar application) is liquid, the liquid composition may be a suspension, a dispersion, a slurry, a solution in a liquid carrier selected from water, organic solvents oils or a mixture thereof. The liquid composition may be prepared by mixing the guar derivatives as described above with the liquid carrier, optionally with other components, by using conventional methods. Preferably, the liquid composition is in the form of an aqueous solution. The composition may comprise from 1 wt % to 60 wt % of the guar derivative based on the total weight of the composition. Preferably, the composition comprises from 5 wt % to 35 wt % of the guar derivative based on the total weight of the composition. In some aspects, the composition comprises from 20 wt % to 30 wt % of the guar derivative based on the total weight of the composition. When conducting seed treatment in industrial scale, it is preferred that the liquid composition used for the seed treatment contains high concentration of the guar derivative, so that less volume of the liquid composition is required to achieve the desired dosage for the treatment (i.e. the weight ratio of the guar derivative to the seeds being treated). Using small volume of the liquid composition can save costs and is less tedious. However, when the concentration of the guar derivative in the liquid composition increases, the fluidity of the liquid composition will significantly decrease. As a result, the liquid composition may become too “thick” to be effectively applied to the seed or the soil, and has poor ability to spread on the surface of the seed or in the soil as well. For example, an aqueous composition comprising 3 wt % of a high molecular weight guar derivative may already be very thick and thus have poor fluidity. One advantage of the present invention is that the guar derivative according to the present invention may have relatively low molecular weight. In such case, the resulting liquid composition can maintain excellent fluidity even if the guar derivative is present at high concentrations, and therefore, such liquid composition can be conveniently used for treating the seeds or the soil. In one embodiment, the method of the present invention comprises a step in which the seed is coated with the composition as described above. Then the coated seed may be applied onto or in the soil, notably, in order to set in contact the coated seed with the ground.

Suitable coating techniques may be utilized to coat the seed or agglomeration of the seeds with the composition according to the present invention. Equipment that may be utilized for coating can include but are not limited to drum coaters, rotary coaters, tumbling drums, fluidized beds and spouted beds. It is appreciated that any suitable equipment or technique known by a person skilled in the art may be employed. The seed may be coated via a batch or continuous coating process. The seed may be coated with the composition according to the present invention which is either in solid form or liquid form. Preferably, an aqueous dispersion or solution is used.

The seeds may be separated prior to the coating step. In one embodiment, mechanical means, such as a sieve, may be employed for separating the seeds. The separated seeds can then be introduced into a coating machine having a seed reservoir. In one embodiment, the seeds are combined with the composition described herein, optionally with a binder and/or adhesive, in a mixing bowl.

In some aspects, one or more layers of coating which comprises the composition according to the present invention may be added onto the seeds or the agglomeration thereof. Outer layers can be introduced sequentially by coating the seeds or the agglomeration thereof in a rotating drum.

Agglomerators or agglomerator devices may also be utilized. Coating may be performed within a rotary coater by placing the seeds within a rotating chamber, which pushes the seeds against the inside wall of the chamber. Centrifugal forces and mixing bars placed inside the coater allow the seeds to rotate and mix with a coating layer comprising the composition according to the present invention. Binder or other coating materials can be pumped into the proximate center of the coater onto an atomizer disk that rotates along with the coating chamber. Upon hitting the atomizer disk, liquid adhesive is then directed outward in small drops onto the seeds.

Seed coating techniques also include, for example, placing the seeds in a rotating pan or drum. The seeds are then mist with water or other liquid, and then gradually a fine inert powder, e.g., diatomaceous earth, is added to the coating pan. Each misted seed becomes the center of a mass of powder, layers, or coatings that gradually increases in size. The mass is then rounded and smoothed by the tumbling action in the pan, similar to pebbles on the beach. The coating layers are compacted by compression from the weight of material in the pan. Binders often are incorporated near the end of the coating process to harden the outer layer of the mass. Binders can also reduce the amount of dust produced by the finished product in handling, shipping and sowing. Screening techniques, such as frequent hand screening, are often times utilized to eliminate blanks or doubles, and to ensure uniform size. For example, tolerance for seed coating compositions described herein can be +/− 1/64 inch (0.4 mm), which is the US seed trade standard for sizing, established long before coatings were introduced. For example, coated lettuce seed is sown most frequently with a belt planter through an 8/64 inch (3.2 mm) diameter round holes in the belt. This hole size requires that the lettuce seeds coated with the composition according to the present invention can be sized over a 7.5/64 inch (3.0 mm) screen and through an 8.5/64 inch (3.4 mm) screen.

In one embodiment of the present invention, the seed may be contacted with the composition by using an “in situ coating” process, notably by implanting in a hole or a furrow in the soil a seed of a plant, and then applying the composition according to the present invention to surround or partially surround, or to be adjacent to the seed, so that the seed come into contact with the composition, notably with the guar derivative. According to the invention, the hole may notably be a hole, a cavity or a hollowed area. The seed may be one that has not be treated by any agent, or a seed that has been treated with an agrochemical (such as fungicide and insecticide) and that has not been treated with the composition of the present invention. Preferably, the composition is deposited on the carrier to provide a granule or a micro granule before being applied. The granule or the micro granule containing the guar derivative may be prepared by using the methods described above.

In still another embodiment, the guar derivative according to the present invention (or the composition containing said guar derivative) is administered to a soil in which a plant is cultivated. Then the seeds of the plant can be applied to the soil so that the seeds will come into contact with the composition, notably with the guar derivative. Notably, the composition in liquid form, such as in the form of aqueous solution/dispersion, or the composition in solid form, such as in powder or granule, may be used.

Preferably, the application of the seed and the application of the composition according to the present invention are performed mechanically. It is appreciated that either or both of the referenced applications can be performed manually as well.

According to a preferred embodiment, the guar derivative as defined above is used in a liquid form.

In one embodiment of the present invention, the guar derivative is used in an amount ranging from 50 to 500 g/quintal seed.

### Biofungicide

The term “bio fungicide” as used herein means a component controlling or eliminating the fungal activity by biological means, such as by using a microorganism such as bacterium, as opposed to the use of a synthetic chemical agent.

By “microorganism” is meant herein a microscopic organism, which may exist in its single-cell form or as colony of cells. In a particular embodiment, said microorganism is unicellular.

The present invention relates more particularly to soil microorganisms, also known as soil microbes.

According to an embodiment, the microorganisms are fungi, in particular unicellular fungi, or bacteria. In a particular embodiment, the microorganisms are bacteria.

According to an embodiment, the biofungicide of the invention is a suppressive microorganism, that is to say a microorganism having a suppressive action on pathogenic fungi.

“Suppressive microorganism” as used herein means any microorganism that can kill or inhibit the growth of fungi by any means. Suppressive microorganism, for instance suppressive bacteria, can be selected that inhibit or kill pathogenic fungi in numerous ways including changing the pH of the microenvironment to one that inhibits or kills the fungi, and producing harmful byproducts like hydrogen cyanide, antifungal substances, cell-wall degrading (lytic) enzymes, and iron chelating compounds called siderophores.

According to an embodiment, the bacteria according to the invention are chosen from Gram-positive bacteria.

As used herein, the term “gram-positive bacteria” refers to bacterial cells which stain violet (positive) in the Gram stain assay. The Gram stain binds peptidoglycan which is abundant in the cell wall of gram-positive bacteria. In contrast, the cell wall of “gram-negative bacteria” has a thin layer of peptidoglycan, thus gram-negative bacteria do not retain the stain and allow to uptake the counterstain in the Gram stain assay.

Gram-positive bacteria are well-known from the skilled person and include bacteria from the Actinobaculum, Actinomyces, Arthrobacter, Bifidobacterium, Frankia, Gardnerella, Lysinibacillus, Microbacterium, Micrococcus, Micromonospora, Mycobacterium, Nocardia, Rhodococcus, Streptomyces, Bacillus, Clostridium, Listeria, Enterococcus, Lactobacillus, Leuconostoc, Mycoplasma, Ureaplasma, Lactococcus, Paenibacillus, Pediococcus, Acetobacterium, Eubacterium, Heliobacterium, Heliospirillum and Sporomusa genera.

In a particular embodiment, the Gram-positive bacteria are selected from the group consisting in Streptomyces and Bacillus, genera bacteria.

In a particular embodiment, the Gram-positive bacteria are bacteria from the Bacillus genera, in particular bacteria selected from the group consisting of Bacillus pumilus (such as B. pumilus strain GB34 (YieldShield; Bayer), B. pumilus strain QST2808 (Sonata; Bayer) and B. pumilus strain BU F-33), Bacillus firmus (such as B. firmus strain 1-1582 (Votivo and Nortica; Bayer)), Bacillus subtilis (such as B. subtilis strains GB03 (Kodiak; Bayer), MBI 600 (Subtilex; Becker Underwood) and QST 713 (Serenade; Bayer), B. subtilis strain GB122 plus, B. subtilis strain EB120, B. subtilis strain J-P13, B. subtilis FB17, B. subtilis strains QST30002 and QST3004 (NRRL B-50421 and NRRLB-50455), B. subtilis strains QST30002 and QST3004 (NRRL B-50421 and NRRLB-50455) sandpaper mutants, B. subtilis strain QST 713, B. subtilis strain DSM 17231, B. subtilis strain KAS-001, B. subtilis strain KAS-006, B. subtilis strain KAS-009, B. subtilis strain KAS-010, B. subtilis strain KAS-011 and B. subtilis strain CCT0089), Bacillus thuringiensis (such as B. thuringiensis galleriae strain SDS-502, B. thuringiensis kurstaki VBTS 2546, B. thuringiensis subsp. kurstaki strain SA 11, B. thuringiensis subsp. kurstaki strain SA 12, B. thuringiensis subsp. kurstaki strain ABTS 351, B. thuringiensis subsp. kurstaki strain EG 2348, B. thuringiensis subsp. kurstaki strain VBTS 2477 quadruple enterotoxin-deficient mutants, B. thuringiensis subsp. aizawai strain GC 91, and B. thuringiensis subsp. tenebrionis), or bacteria from the Streptomyces genera, in particular bacteria from the Streptomyces K61 species.

In a more particular embodiment, the Gram-positive bacteria are bacteria from the B. subtilis, the B. thuringiensis or the B. megaterium species. In still a particular embodiment, the Gram-positive bacteria are B. subtilis CCT 0089, B. thuringiensis CCT 2335 or B. megaterium CCT 0536.

According to an embodiment, the bacteria according to the invention are chosen from Gram-negative bacteria.

Gram-negative bacteria are well-known from the skilled person and include bacteria from the Acetobacter, Achromobacter, Actinobacillus, Agrobacterium, Allorhizobium, Azospirillum, Azotobacter, Bordetella, Bradyrhizobium, Brucella, Burkholderia, Campylobacter, Carbophilus, Chelatobacter, Chryseobacterium, Citrobacter, Delftia, Enterobacter, Erwinia, Escherichia, Flavobacterium, Francisella, Frateuria, Gluconobacter, Helicobacter, Haemophilus, Kalstia, Klebsiella, Legionella, Mesorhizobium, Moraxella, Neisseria, Pantoea, Pasteurella, Phyllobacterium, Proteus, Pseudomonas, Rhizobium, Salmonella, Serratia, Shigella, Sinorhizobium, Treponema, Vibrio, Xanthomonas and Yersinia genera.

In a particular embodiment, the Gram-negative bacteria are selected from the group consisting in Pseudomonas genera bacteria.

In a particular embodiment, the Gram-negative bacteria are bacteria from the Acetobacter genera, in particular bacteria from the Pseudomonas genera, in particular bacteria selected from the group consisting in Pseudomonas putida, Pseudomonas fluorescens, Pseudomonas protegens, Pseudomonas chlororaphis (such as Pseudomonas chlororaphis strain MA342), Pseudomonas aurantiaca, Pseudomonas mendocina and Pseudomonas rathonis species.

In more particular embodiments, the Gram-negative bacteria are bacteria from the B. japonicum or the P. putida species. In still a particular embodiment, the Gram-negative bacteria are B. japonicum strain CCT 4065 or P. putida CCT 5357.

According to anyone of the invention embodiments, the microorganism may be for instance bacteria chosen from the B. subtilis, the B. megaterium, the B. thuringiensis, the B. japonicum or the P. putida species.

According to an embodiment, the microorganims according to the invention are fungi, in particular unicellular fungi.

Fungi are well-known from the skilled person and include Ascomycetes, Glomeromycetes and Basidiomycetes. In a particular embodiment, said fungi are selected from the Ascomycetes phylum, in particular from the group consisting in the Trichoderma, Metarhizium, Beauveria, Lecanicillium, Purpureocillium, Gliocladium, Isaria, Fusarium, Arthrobotrys, Penicillium, Aspergillus, Ampelomyces, Coniothyrium, Aureobasidium and Candida genera; from the Glomeromycetes phylum, in particular from the group consisting in the Glomus and Rhizophagus genera; and/or from the Basidiomycetes phylum, in particular from the group consisting in the Phlebiopsis and Rhizoctonia genera.

In a particular embodiment, said fungi are fungi from the Trichoderma genera, in particular fungi selected from the group consisting in the Trichoderma viride, Trichoderma atroviride (such as Trichoderma atroviride strain 1-1237, Trichoderma atroviride strain SC1, Trichoderma atroviride strain Trichoderma harzianum (such as, Trichoderma harzianum Rifai strain T-22 and ITEM-908), Trichoderma asperellum (such as Trichoderma asperellum strain ICC012 T25 and TV1, Trichoderma asperellum strain T34),; fungi from the Metarhizium genera, in particular fungi selected from the group consisting in the Metarhizium anisopliae, such as Metarhizium anisopliae var. anisopliae BIPESCO 5/F52,; fungi from the Beauveria genera, in particular fungi from the Beauveria bassiana species (such as Beauveria bassiana strain ATCC 74040, Beauveria bassiana strain GHA, Beauveria bassiana strain NPP111B005, and Beauveria bassiana strain 147); fungi from the Lecanicillium genera, in particular fungi selected from Lecanicillium muscarium species, such as Lecanicillium muscarium strain Ve6; fungi from the Gliocladium genera, in particular fungi from the Gliocladium catenulatum species, such as Gliocladium catenulatum strain J1446; fungi from the Isaria genera, in particular fungi from the Isaria fumosorosea species, such as Isaria fumosorosea Apopka strain 97; fungi from the Ampelomyces genera, in particular fungi from the Ampelomyces quisqualis species; fungi from the Candida genera, in particular fungi from the Candida oleophila species; fungi from the Phlebiopsis genera, in particular fungi from the Phlebiopsis gigantea species.

In more particular embodiments, the fungi are fungi from the Trichoderma harzianum or Beauveria bassiana species. In still a particular embodiment, the fungi are Trichoderma harzianum CCT 4790 or Beauveria bassiana ATCC 7159/DSM 1344.

According to anyone of the invention embodiments, the microorganism may be for instance bacteria chosen from the B. subtilis, the B. megaterium, the B. thuringiensis, the B. japonicum or the P. putida species or fungi from the T. harzianum or B. bassiana species, such as those described previously.

The amount of microorganism to be used may vary from one microorganism to another and may also depend on the seed to be treated. In one embodiment of the present invention, the microorganism is used in an amount ranging from 1.10⁴ to 1.10¹⁶ CFU/quintal seed.

The present invention also relates to a method for maintaining or increasing the growth rate and/or biofungicidal activity of such microorganisms, in particular of suppressive microorganism, comprising a step of contacting at least one seed with a guar derivative as defined above.

According to a preferred embodiment, this method is carried out in liquid medium. Therefore, preferably, this method comprises a step of contacting at least one seed with a guar derivative as defined above in a liquid form or with a liquid composition comprising a guar derivative as defined above.

The present invention also relates to the use of a microorganism, in particular a suppressive microorganism, and of a guar derivative as defined above, as bio fungicide.

Therefore, the present invention relates to the combined use of said microorganism, in particular suppressive microorganism, and guar derivative. It has been shown that the combination of said microorganism, in particular suppressive microorganism, and guar derivative gives a bio fungicide activity.

The present invention also relates to a biofungicide composition comprising at least one microorganism, in particular a suppressive microorganism, and at least one guar derivative as defined above.

According to anyone of the invention embodiments, the microorganism and the guar derivative are combined in a ratio microorganism:guar derivative ranging from 1.10⁴ to 1.10¹⁵, for example ranging from 1.10⁴ to 1.10¹², for example ranging from 1.10⁴ to 1.10¹¹ CFU/g, for example ranging from 1.10⁴ to 5.10¹⁰ CFU/g, for example ranging from 1.10⁵ to 1.10¹⁰ CFU/g. For instance, the microorganism and the guar derivative may be combined in a ratio microorganism: guar derivative ranging from 1.10⁸ to 1.10¹².

Preferably, this bio fungicide composition is in a liquid form.

The present invention also relates to a kit comprising at least one microorganism, in particular a suppressive microorganism, and at least one guar derivative as defined above, said kit being preferably used as biofungicide.

The present invention thus also relates to the use of the above-mentioned kit as bio fungicide.

The present invention also relates to a seed coated with the biofungicide composition as defined above.

In one embodiment, the seed is of the crop or plant species including but not limited to corn (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus animus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, woody plants such as conifers and deciduous trees, squash, pumpkin, hemp, zucchini, apple, pear, quince, melon, plum, cherry, peach, nectarine, apricot, strawberry, grape, raspberry, blackberry, soybean, sorghum, sugarcane, rapeseed, clover, carrot, and Arabidopsis thaliana.

In one embodiment, the seed is of any vegetables species including but not limited to tomatoes (Lycopersicon esculentum), lettuce (e.g., Lactuca sativa), green beans (Phaseolus vulgaris), lima beans (Phaseolus limensis), peas (Lathyrus spp.), cauliflower, broccoli, turnip, radish, spinach, asparagus, onion, garlic, pepper, celery, and members of the genus Cucumis such as cucumber (C. sativus), cantaloupe (C. cantalupensis), and musk melon (C. melo).

In one embodiment, the seed is of any ornamentals species including but not limited to hydrangea (Macrophylla hydrangea), hibiscus (Hibiscus rosasanensis), petunias (Petunia hybrida), roses (Rosa spp.), azalea (Rhododendron spp.), tulips (Tulipa spp.), daffodils (Narcissus spp.), carnation (Dianthus caryophyllus), poinsettia (Euphorbia pulchenima), and chrysanthemum.

In one embodiment, the seed is of any conifer species including but not limited to conifers pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In one embodiment, the seed is of any leguminous plant species including but not limited beans and peas. Beans include guar, locust bean, fenugreek, soybean, garden beans, cowpea, mungbean, lima bean, fava bean, lentils, chickpea, pea, moth bean, broad bean, kidney bean, lentil, dry bean, etc. Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Typical forage and turf grass for use in the methods described herein include but are not limited to alfalfa, orchard grass, tall fescue, perennial ryegrass, creeping bent grass, lucerne, birdsfoot trefoil, clover, stylosanthes species, lotononis bainessii, sainfoin and redtop. Other grass species include barley, wheat, oat, rye, orchard grass, guinea grass, sorghum or turf grass plant.

In another embodiment, the seed is selected from the following crops or vegetables: corn, wheat, sorghum, soybean, tomato, cauliflower, radish, cabbage, canola, lettuce, rye grass, grass, rice, cotton, sunflower and the like. In another embodiment, the seed is selected from corn, wheat, barley, rice, peas, oats, soybean, sunflower, alfalfa, sorghum, rapeseed, sugar beet, cotton, tobacco, forage crops, linseed, hemp, grass, vegetables, fruits and flowers seeds.

It is understood that the term “seed” or “seedling” is not limited to a specific or particular type of species or seed. The term “seed” or “seedling” can refer to seed from a single plant species, a mixture of seed from multiple plant species, or a seed blend from various strains within a plant species. In one embodiment, crop seeds include but are not limited to rice, corn, wheat, barley, oats, soybean, cotton, sunflower, alfalfa, sorghum, rapeseed, sugarbeet, tomato, bean, carrot, tobacco or flower seeds.

An aspect of the invention includes methods of using the bio fungicide composition of the invention to control, prevent or reduce pathogenic fungus infestations in growing plants, on seeds, and on harvested crops.

A biofungicide composition of the invention can be used to treat plants, seeds, plants, leaves, cuttings, and plant media and for post-harvest treatment of crops.

As mentioned previously, the present invention also relates to a bio fungicide composition comprising at least one microorganism, in particular a suppressive microorganism, and at least one guar derivative as defined above.

According to a preferred embodiment, the composition is applied onto the foliar system of the plant. Such application is preferably carried out by spraying a composition as disclosed above onto the leaves of the plant. For example, the composition can be sprayed onto a field using appropriate means well known in agriculture.

For instance, a bio fungicide composition of the present invention may be dilute enough to be readily sprayed using standard agricultural spray equipment. Application of the composition to foliage may be accomplished by spraying, using any conventional means for spraying liquids, such as spray nozzles, atomizers or the like. The composition of the invention can be used in precision farming techniques, in which apparatus is employed to vary the amount of pesticide applied to different parts of a field, depending on variables such as the particular plant species present, soil composition, etc. In one embodiment of such techniques, a global positioning system operated with the spraying apparatus can be used to apply the desired amount of the composition to different parts of a field. The selection of application rates that are effective for a composition of the invention is within the skill of the ordinary agricultural scientist.

According to a preferred embodiment, the present invention relates to a method for treating a plant wherein a composition as defined previously is applied onto at least one part of said plant.

The composition may be applied directly onto the plant, or may be diluted just before application with a liquid diluent comprising water or a mixture of water and organic solvent, or may be mixed just before application with another agrochemical composition.

In one embodiment, the composition may be applied onto the foliar system of the plant, preferably by spraying said composition onto the leaves of the plant.

The present invention is not limited to bio fungicide compositions but also relates to any microorganism useful in agricultural applications, such as biopesticides and the like.

The following examples are included to illustrate embodiments of the invention, but is not limited to described examples.

### EXAMPLES

### Example 1

The following materials are used in the experiments:

Guar: Cyamopsis tetragonoloba (guar) gum, available from Solvay (provided as a powder)

Bacteria strains were acquired from Tropical Culture Collection in André Tosello Foundation—Brazil.
Bacillus subtilis CCT 0089
Bacillus megaterium CCT 0536
Bradyrhizobium japonicum CCT 4065

All strains were stored at −80° C. in the appropriated culture media, containing 15% of glycerol.

Two different culture media were used in the experiments:
NA media containing per liter: 3 g of meat extract, 5 g of peptone and 15 g of agar (for solid media only)
YMA media containing per liter: 0.5 g of monobasic potassium phosphate, 0.2 g of magnesium sulphate; 0.1 g of sodium chloride; 0.5 g of yeast extract; 10 g of mannitol (for inoculum and solid media only); 5 mL of a 5% bromothymol blue solution and 15 g of agar (solid media only).

For strains Bacillus subtilis and Bacillus megaterium NA media was used. For strain Bradyrhyzobium japonicum, YMA media was used. These media were selected according to strains supplier.

A 250 mL shake flask containing 100 mL of NA or YMA culture media, was inoculated with 1 mL of the stock culture and incubated at 30° C., 150 rpm for 72 hours.

For each strain, 10 mL of the reactivation media were then transferred into a 250 mL shake flask containing 100 mL of the same media, with the addition of guar powder (at 0.7 wt % in the incubation media); and incubated at 30° C., 150 rpm, for 96 hours. An experiment without addition of guar powder is also performed for each strain as a control.

100 μL samples of each experiments were taken after 0 h, 24 h, 48 h, 72 h and 96 h of incubation. These samples were diluted (the dilutions were variable according to strain growth, being from 1×10⁻⁵ up to 1×10⁻¹⁵) and the dilutions plated in solid NA or YMA media. The plates were incubated at 30° C. until appearance of colonies. After incubation, the number of colonies present in each dilution was counted and used to evaluate bacterial growth.

For bacterial growth rate determination, a graph of the log₁₀ (number of colonies) versus time of incubation was constructed. The straight line in this graph represents the exponential phase of bacterial growth and the angular coefficient represents the bacterial growth rate (μ).

The μ value was used to compare all the experiments and to evaluate the influence of guar addition on bacterial growth.

### Example 1a

In a first set of experiments the ratio of microorganisms and guar is equal to 1.00×10⁶ CFU/g. The bacteria growth rate (μ) obtained for the different experiments are summarized in Table 1a:.

TABLE 1a

| | Bacteria growth |
|---|---|
| Composition | rate (h⁻¹) |
| Bacillus subtilis CCT 0089 | 0.0647 |
| Bacillus subtilis CCT 0089 + guar | 0.0657 |
| Bacillus megaterium CCT 0536 | 0.0605 |
| Bacillus megaterium CCT 0536 + guar | 0.0878 |
| Bradyrhyzobium japonicum CCT 4065 | 0.0891 |
| Bradyrhyzobium japonicum CCT 4065 + guar | 0.0939 |

For the three strains, a higher value of bacteria growth rate is obtained in presence of guar. The addition of guar permits to increase the growth rate of these different strains of bacteria. In Table 2a are reported the relative increase of bacteria growth rate with the addition of guar compared to the control for each strain. An increase of bacteria growth rate between 2 and 45% is observed for the two gram positive bacteria (Bacillus subtilis and Bacillus megaterium), whereas a relative increase between 5% is observed for the gram negative bacteria (Bradyrhyzobium japonicum).

TABLE 2a

| | Relative increase of bacteria |
|---|---|
| Strain | growth rate with guar addition |
| Bacillus subtilis CCT 0089 | 2% |
| Bacillus megaterium CCT 0536 | 45% |
| Bradyrhyzobium japonicum CCT 4065 | 5% |

### Example 1b

Another set of experiments was carried out, in which the ratio of microorganisms and guar was equal to 1.0×10¹⁰ CFU/g.

The bacteria growth rate (μ) obtained for the different experiments are summarized in Table 1b:

TABLE 1b

| | Bacteria growth |
|---|---|
| Composition | rate (h⁻¹) |
| Bacillus subtilis CCT 0089 | 0.0862 |
| Bacillus subtilis CCT 0089 + guar | 0.1172 |
| Bacillus megaterium CCT 0536 | 0.0835 |
| Bacillus megaterium CCT 0536 + guar | 0.0912 |
| Bradyrhyzobium japonicum CCT 4065 | 0.0915 |
| Bradyrhyzobium japonicum CCT 4065 + guar | 0.0897 |

For the three strains, a higher or comparable value of bacteria growth rate is obtained in presence of guar. For two of the bacteria strains, the addition of guar permits to increase the growth rate. In Table 2b are reported the relative increase of bacteria growth rate with the addition of guar compared to the control for each strain. An increase of bacteria growth rate between 9 and 36% is observed for the two gram positive bacteria (Bacillus subtilis and Bacillus megaterium). For Bradyrhyzobium Japonicum, a comparable growth rate is obtained with the addition of guar compared to control, hence the addition of guar maintains the growth rate of microorganisms.

TABLE 2b

| | Relative increase of bacteria |
|---|---|
| Strain | growth rate with guar addition |
| Bacillus subtilis CCT 0089 | 36% |
| Bacillus megaterium CCT 0536 | 9% |
| Bradyrhyzobium japonicum CCT 4065 | ~0% |

### Example 2

The Following Materials are used in the Experiments:

Guar: Cyamopsis tetragonoloba (guar) gum, available from Solvay (provided as a powder)

All microorganisms strains were acquired from Tropical Culture Collection in Andrë Tosello Foundation—Brazil, some of them have reference in American Type Culture Colection (ATCC).
Trichoderma harzianum CCT 4790
Beauveria bassiana ATCC 7159/DSM 1344

All strains were stored at −80° C. in the appropriate culture media, containing 20% of glycerol.

Culture Media used in the Experiments:
Nutrient broth (NA) media containing per liter: 3 g of meat extract, 5 g of peptone and 15 g of agar (for solid media only)
Oatmeal Agar (OA) containing per liter: 25 g of oat flakes or flour and 15 g of agar
Malt Extract Agar 2% (MA2) containing per liter: 20 g of malt extract and 15 g of agar

The media OA and MA2 were used for reactivation of the strains T. harzianum and B. bassiana, respectively, according to supplier's recommendation.

For the experiments with guar, only NA media was used.

### Reactivation of Microorganisms:

A Petri dish containing 20 mL of OA or MA2 media was used for the reactivation of the strains T. harzianum and B. bassiana, respectively.

The stock culture was used to inoculate the solid media for each strain and the petri dishes were incubated at 25° C. until complete growth.

Incubation with Guar:

From the reactivation media on petri dish, the spores of fungi were recovered and a spore solution was prepared.

500 μL of the spore solution (approximately 1×10¹⁰ CFU/mL) were transferred to Erlenmeyer flasks containing 50 mL of media (controls and NA media with guar) and incubated at 25° C. Samples were taken at 48 h, 120 h and 168 h, filtered on filter paper and incubated at 60° C. before weighing
Control media=NA without guar addition

### Growth Evaluation:

The dry biomass recovery after each sample was plotted in a graphic dry biomass vs time and the growth curve could be obtained.

The growth rate (μ) was calculated considering only the exponential phase of the growth and compared with the control.

The μ value was used to compare all the experiments and to evaluate the influence of guar addition on fungi growth. The microorganisms growth rate (μ) obtained for the different experiments are summarized in Table 3:

TABLE 3

| | Fungi growth |
|---|---|
| Composition | rate (h⁻¹) |
| Trichoderma harzianum CCT 4790 | 0.0009 |
| Trichoderma harzianum CCT 4790 + guar | 0.0022 |
| Beauveria bassiana ATCC 7159/DSM 1344 | 0.0870 |
| Beauveria bassiana ATCC 7159/DSM 1344 + guar | 0.1120 |

For the two strains, a higher growth rate is obtained in presence of guar. The addition of guar permits to increase the growth rate of these different strains of fungi. In Table 4 are reported the relative increase of fungi growth rate with the addition of guar compared to the control for each strain. An increase of fungi growth rate between 29 and 144% is observed for the two strains of fungi.

TABLE 4

| | Relative increase of fungi growth |
|---|---|
| Strain | rate with guar addition |
| Trichoderma harzianum CCT 4790 | 144% |
| Beauveria bassiana ATCC 7159/ | 29% |
| DSM 1344 | |

### Example 3

### The Following Materials are Used in the Experiments:

Guar: Cyamopsis tetragonoloba (guar) gum, available from Solvay (provided as a powder)

Bacteria strains were acquired from Tropical Culture Collection in André Tosello Foundation—Brazil.
Bacillus thuringiensis CCT 2335
Pseudomonas putida CCT 5357

All strains were stored at −80° C. in the appropriate culture media, containing 15% of glycerol.

Only one culture media was used for both strains
NA media containing per liter: 3 g of meat extract, 5 g of peptone and 15 g of agar (for solid media only)

A 250 mL shake flask containing 100 mL of NA culture media (reactivation media), was inoculated with 1 mL of the stock culture and incubated at 30° C., 150 rpm for 72 hours.

10 mL of this reactivation media were then transferred into a 250 mL shake flask containing 100 ml of culture media, with the addition of guar powder and incubated at 30° C., 150 rpm, for 96 hours.

An experiment without addition of guar powder is also performed for each strain as a control.

100 μL samples of each experiment were taken after 0 h, 24 h, 48 h, 72 h and 96 h of incubation.

These samples were diluted (the dilutions were variable according to strain growth, being from 1×10⁻⁵ up to 1×10⁻¹⁵) and the dilutions plated in solid NA media. The plates were incubated at 30° C. until appearance of colonies. After incubation, the number of colonies present in each dilution was counted and used to evaluate bacterial growth.

For bacterial growth rate determination, a graph of the log₁₀ (number of colonies) versus time of incubation was constructed. The straight line in this graph represents the exponential phase of bacterial growth and the angular coefficient represents the bacterial growth rate (μ).

The μ value was used to compare all the experiments and to evaluate the influence of guar addition on bacterial growth. For this set of experiments the ratio of microorganisms and guar is equal to 1.0×10⁵ CFU/g. The bacteria growth rate (μ) obtained for the different experiments are summarized in Table 5:

TABLE 5

| | Bacteria growth |
|---|---|
| Composition | rate (h⁻¹) |
| Bacillus thuringiensis CCT 2335 | 0.0898 |
| Bacillus thuringiensis CCT 2335 + guar | 0.1047 |
| Pseudomonas putida CCT 5357 | 0.1133 |
| Pseudomonas putida CCT 5357 + guar | 0.1330 |

For the two strains, a higher value of bacteria growth rate is obtained in the presence of guar. Hence, the addition of guar permits to increase the bacteria growth rate. In Table 6 are reported the relative increase of bacteria growth rate with the addition of guar compared to the control for each strain. An increase of bacteria growth rate equals to 17% is observed for the two strains of bacteria.

TABLE 6

| | Relative increase of bacteria growth |
|---|---|
| Strain | rate with guar addition |
| Bacillus thuringiensis CCT 2335 | 17% |
| Pseudomonas putida CCT 5357 | 17% |

1. A fungicide composition comprising a bio-fungicide and a non-derivatized guar gum.

2. The fungicide composition of claim 1, wherein the bio-fungicide is a microorganism.

3. A method for maintaining or increasing the growth rate of microorganisms, comprising a step of contacting said microorganism with a non-derivatized guar gum.

4. (canceled)

5. A kit comprising at least one microorganism and at least one non-derivatized guar gum.

6. (canceled)

7. A method for controlling fungal organism comprising applying a fungicide composition according to claim 1.

8. A method of controlling or preventing infection of a plant by phytopathogenic fungi, comprising the step of applying to said plant a fungicide composition according to claim 1.

9. A method for treating a plant wherein a composition according to claim 1 is applied onto at least one part of said plant.

10. The method of claim 9, wherein the composition is applied onto the foliar system of the plant.

11. The fungicide composition of claim 2, wherein the microorganism is fungi or bacteria.

12. The method of claim 3, wherein the microorganisms are suppressive microorganisms.

13. The kit of claim 5, wherein the at least one microorganism is a suppressive microorganism.

14. The method of claim 10, wherein the composition is applied onto the foliar system of the plant by spraying said composition onto the leaves of the plant.
